(19) United States
(12) Patent Application Publication
(10) Pub. No.: US 2026/0053661 A1
(43) Pub. Date: Feb. 26, 2026

(54) EXTERNAL URINE COLLECTION DEVICE

(71) Applicant: SBE Medical Inc., Belmont, MI (US)

(72) Inventors: Daniel E. Byers, Belmont, MI (US); Nicholas Borgdorff, Caledonia, MI (US); Evan James Bauer, Rockford, MI (US)

(21) Appl. No.: 18/812,336

(22) Filed: Aug. 22, 2024

(51) Int. Cl. A61F 5/455; A61F 5/443; A61F 5/453

(52) U.S. Cl. CPC A61F 5/4556; A61F 5/443; A61F 5/453

(57) ABSTRACT

An external urine collection device for collecting urine excreted by a user through the user's external urethral orifice includes a sleeve and a cup that defines a collection chamber and a urine outlet. The sleeve can be attached to an area around the user's external urethral orifice by an adhesive. The cup can be releasably coupled to the sleeve. Pull tabs on the cup and sleeve can be gipped and pulled apart to separate the cup from the sleeve. The sleeve can include a finger gap so that a caregiver can hold the sleeve securely during installation of the sleeve, assembly of the cup, and detachment of the cup from the sleeve. The cup can include a site window for viewing the user's external urethral orifice and surrounding anatomy without removing the cup. A related system, method of assembly, and method of use is provided.

### FIELD OF THE INVENTION

The present embodiments generally relate to an external urine collection device and urine collection system, and related methods of assembly and use.

### BACKGROUND OF THE INVENTION

Hospital patients experiencing urinary incontinence are traditionally treated using indwelling catheters. Inserting the catheter can be an invasive and time consuming procedure, as it requires the use of sterile practices. Inserting the catheter can be particularly challenging with elderly patients and patients who are overweight. Indwelling catheters can increase a patient's chance of developing a urinary tract infection (UTI), which requires treatment. UTIs can be painful for the patient and treatment of the infection can sometimes delay discharging the patient from the hospital. In addition, when the UTI is caused by the hospital's indwelling catheter, the hospital is often responsible for payment of the treatment, which can consume hospital resources that otherwise could be expended on improving healthcare.

There are several alternatives to indwelling catheters that do not require a component to be inserted into the patient's urethra. For example, one type of alternative includes a bag or container that is adhesively attached to the patient's body. However, this type of device can be difficult to remove without causing discomfort to the patient, and does not allow quick access to the patient's urethra and surrounding site.

Another alternative device has a two-piece design, with a funnel detachable from a sleeve adhesively attached to the patient's body. While this two-piece design does allow for some access to the site without fully moving the device, the funnel sometimes can be difficult to detach and reattach.

While various efforts have been made, there remains an opportunity to provide additional systems and methods for collecting urine excreted by a patient using a device that does not require insertion of components into the urethra of the patient, does not cause discomfort to the patient during removal, and does not restrict quick access to the patient's urethra and surrounding anatomy.

### SUMMARY OF THE INVENTION

An external urine collection device for collecting urine excreted by a user through the user's external urethral orifice is provided. A system, method of assembly, and method of use of the device are also provided.

In one embodiment, an external urine collection device can include a posterior end and an anterior end, a sleeve having a sleeve outlet and an interface surface defining a sleeve inlet, the interface surface configured to be secured via an adhesive to an external surface of the user and to surround an external urethral orifice of the user, and a cup coupled with the sleeve at the sleeve outlet. The cup includes a collection chamber and a urine outlet.

In another embodiment, the device can include a releasable coupling between the cup and the sleeve. The releasable coupling can form a liquid tight seal for the flow of urine through the sleeve and into the cup.

In still another embodiment, a first pull tab can be joined with the sleeve at the anterior end of the device, and a second pull tab can be joined with the cup at the anterior end of the device. The cup can be releasable from the sleeve while the sleeve remains secured via the adhesive to the external surface of the user by a caregiver gripping the first and second pull tabs and pulling the second pull tab away from the first pull tab while the first pull tab remains proximate the user. The pull tabs can reduce the pulling force transmitted from the sleeve to the user during release of the cup.

In another embodiment, the second pull tab can extend anteriorly from the cup in a direction along a sagittal midplane of the cup.

In yet another embodiment, the first pull tab can be transverse to the second pull tab.

In still another embodiment, the device can comprise a sagittal midplane configured to lie substantially within or parallel to a sagittal plane of the user when the collection device is worn by the user. The second pull tab can lie in the sagittal midplane such that the second pull tab is pulled in a direction generally along the sagittal plane of the user to remove the cup from the sleeve.

In a further embodiment, the first pull tab can be spaced from the interface surface in a direction along a vertical axis of the sleeve to define a finger gap between the interface surface and the first pull tab.

In another embodiment, the sleeve and/or the cup can be constructed from a flexible polymeric material.

In yet another embodiment, the sleeve can include a side wall, with the interface surface at a proximal end of the side wall and a flange at a distal end of the side wall. The flange can extend transversely from the side wall and the flange is spaced distally from the interface surface to define a finger gap therebetween.

In another embodiment, the interface surface can have a first length at the anterior end of the sleeve and the first pull tab has a second length at the anterior end of the sleeve that is greater than the first length such that the first pull tab projects beyond the interface surface.

In yet another embodiment, the cup can include a funnel-shaped wall defining the collection chamber.

In still another embodiment, the cup can include a drain connector through which urine can exit the collection chamber. The drain connector can comprise a press fit connector configured to receive the outer diameter of a drain tube.

In a further embodiment, the cup can include a site window between the posterior and anterior ends. The external urethral orifice of the user can be viewed through the site window without removing the cup from the sleeve.

In another embodiment, the releasable coupling can include a male connector disposed on one of the sleeve and the cup and a female connector disposed on the other of the sleeve and the cup. The female connector can be configured to receive the male connector.

In yet another embodiment, a system for collecting urine excreted by a user through the user's external urethral orifice is provided.

In a further embodiment, a method of managing urine excreted by a user through the user's external urethral orifice is provided.

These and other features and advantages of the present invention will become apparent from the following description of the invention, when viewed in accordance with the accompanying drawings and appended claims.

Before the embodiments are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a posterior perspective view of an external urine collection device according to a current embodiment;

FIG. 2 is a side elevational view of the external urine collection device;

FIG. 3 is a top-down view of the external urine collection device;

FIG. 4 is an exploded perspective view of the external urine collection device;

FIG. 5 is a sagittal sectional view of a sleeve for the external urine collection device;

FIG. 6 is a sagittal sectional view of a cup for the external urine collection device;

FIG. 7 is an anterior elevational view of the external urine collection device;

FIG. 8 is a sagittal sectional view of the external urine collection device with the sleeve and cup coupled together;

FIG. 9 is a schematic view of an external urine collection system according to another embodiment; and

FIG. 10 is a schematic view of an external urine collection system according to yet another embodiment.

### DETAILED DESCRIPTION

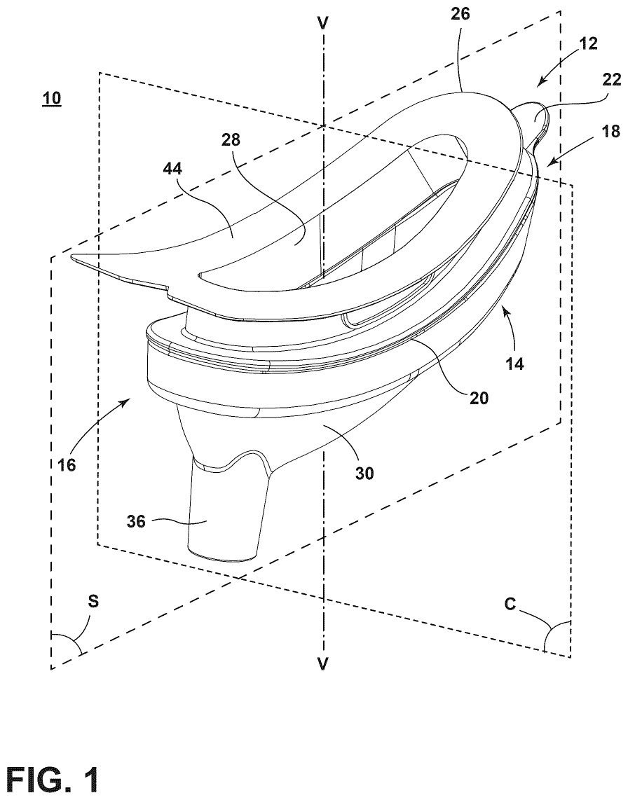

An external urine collection device is illustrated in FIGS. 1-3 and generally designated 10. The external urine collection device 10 is configured to be worn externally by a user, around the user's urethral orifice, to collect urine excreted through the urethral orifice. As used herein, the term “external” when used to describe the urine collection device 10 refers to a device which is adapted to be used without inserting components inside the user's urethra. The urine collection device 10 is optionally part of a urine collection system, as will be described in further detail below, to collect and remove urine excreted from the user. While the collection device 10 is described in the context of collecting urine, it is to be understood that the collection device 10 is not limited to collecting urine but may also collect other liquids excreted through the user's urethral orifice or vaginal orifice in a similar manner.

As used herein, the term “user” refers to the person to which the external urine collection device 10 is administered, i.e., the person wearing the device 10, and the term “caregiver” as used herein refers to one who assists in administration of the device 10 to the user, and includes healthcare professionals or trained professionals include but not limited to doctors, nurses, or non-healthcare professionals such as family members or friends who assist in administration of device 10 to the user. It is contemplated that in some instances the caregiver may be the user themselves.

The external urine collection device 10 includes a ring or sleeve 12 and a cup 14 that can be removably coupled and uncoupled to the sleeve 12. For the purpose of discussion, the collection device 10 may be considered to include a posterior end 16 and an anterior end 18 according to the anatomical orientation of the collection device 10 when worn by a user. When worn by a user, the sleeve 12 is adhered to the user's body around the user's urethral orifice, and the posterior end 16 is positioned posterior of the user's urethral orifice while the anterior end 18 is positioned anterior of the user's urethral orifice.

The collection device 10 has a sagittal midplane S, which is also the sagittal midplane of the sleeve 12 and cup 14 individually. The sagittal midplane S divides the device 10 into left and right. When the collection device 10 worn by a user, the sagittal midplane S lies substantially within a sagittal plane of the user or may be substantially parallel to a sagittal plane of the user. A frontal or coronal plane C of the device 10 is orthogonal to the sagittal midplane S and divides the device 10 into a dorsal portion including the posterior end 16 and a ventral portion including the anterior end 18. When the collection device 10 worn by a user, the coronal plane C may be substantially parallel to a coronal plane of the user. Alternately, the coronal plane C may substantially within or may intersect a coronal plane of the user. A vertical axis V of the collection device 10 is formed by the intersection of the sagittal and coronal planes S, C.

The collection device 10 has a releasable coupling 20 between the cup 14 and the sleeve 12, the releasable coupling 20 forming a liquid tight seal for the flow of urine through the sleeve 12 and into the cup 14. The cup 14 can be installed on or separated from the sleeve 12 while the sleeve 12 remains secured via an adhesive to an external surface of the user.

To reduce forces on the user's body during separation of the cup 14, the collection device 10 includes pull tabs 22, 24. The sleeve 12 comprises a first pull tab 22 and the cup 14 comprises a second pull tab 24. The pull tabs 22, 24 can be provided in various forms, such as a small flap, strap, projection loop, or similar appendage used for pulling, and are configured to be gripped by a caregiver by pinching the tab between the thumb and forefinger. The cup 14 is releasable from the sleeve 12 while the sleeve 12 remains secured via the adhesive to the user by gripping the first and second pull tabs 22, 24 and pulling the second pull tab 24 away from the first pull tab 22 while the first pull tab 22 remains proximate the user. The pull tabs 22, 24 reduce the pulling force transmitted from the sleeve 12 to the user during release of the cup 14.

Still referring to FIGS. 1-3, the sleeve 12 includes an interface surface 26 that is configured to be adhered to the user's body around the user's urethral orifice and to surround an external urethral orifice of the user. The interface surface 26 generally defines an opening or urine inlet 28 that is configured to allow urine or other substances excreted by the user to flow through the sleeve 12 and into the cup 14 when the external urine collection device 10 is worn by the user. The sleeve inlet 28 can be configured to surround the user's external urethral orifice. Optionally, the sleeve inlet 28 is configured to surround the user's labia majora.

The cup 14 includes a wall 30 defining a collection chamber 32 in which urine excreted by the user can be collected during excretion and/or prior to removal. The collection chamber 32 is fluidly connected with an outlet 34 through which urine in the collection chamber 32 can drain from the collection chamber 32 and exit the urinary device 10 through a drain 36.

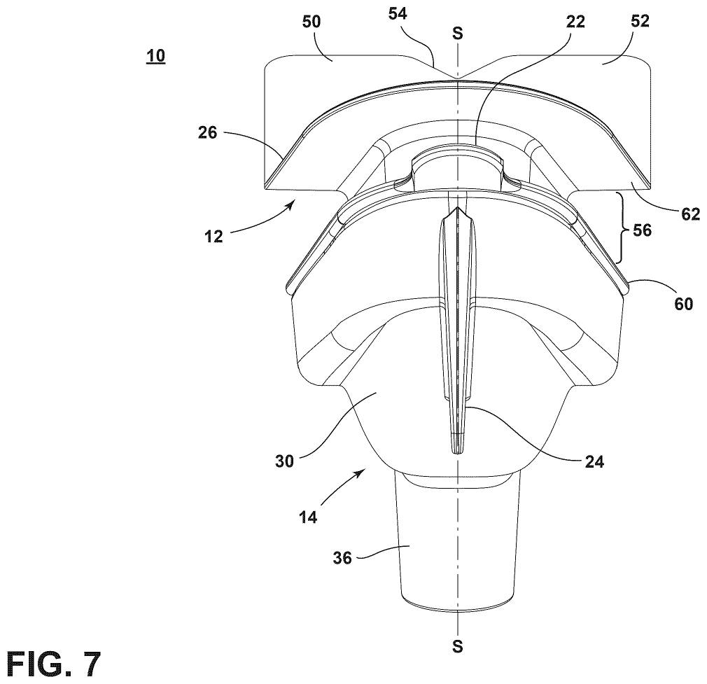

The contours of the wall 30 forming the collection chamber 32 and/or the shape, location, and/or dimensions of the outlet 34 may be selected to provide the desired urine draining characteristics. For example, the wall 30 can comprise a funnel-shaped wall, such that the cup 14 funnels urine away from the user's body and toward the drain 36. The wall 30 can be asymmetrical about the coronal plane C, and can be angled or curved, as illustrated, to encourage urine in the anterior end 18 to flow toward the posterior end 16. The wall 30 can be symmetric about the sagittal plane S, as best seen in FIG. 7, so that urine flows toward the sagittal plane S from the left and right sides of the device 10.

The outlet 34 can be disposed in the wall 30 on the side of the coronal plane C defining the dorsal portion of the device 10 and including the posterior end 16, as illustrated, to facilitate draining of the urine in the collection chamber 32. Optionally, the outlet 34 can be positioned anywhere along the sagittal plane S.

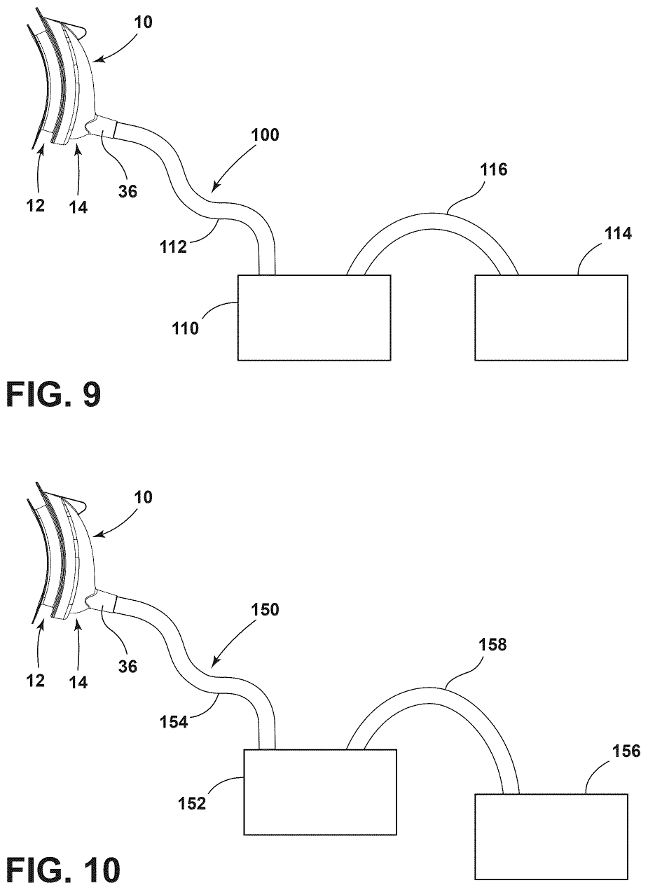

The drain 36 can be in the form of a connector for connecting a drain tube or can comprise a drain tube through which urine can exit the collection chamber 32. In the illustrated embodiment, the drain 36 is a drain connector 36, which can be configured to connect with a drain tube (e.g., drain tube 112 in FIG. 9 or drain tube 154 in FIG. 10) for disposal of the drained urine in a collection container (e.g., container 110 in FIG. 9 or drain container 156 in FIG. 10). Optionally, when the drain 36 includes a drain tube, the drain 36 connects directly with the collection container.

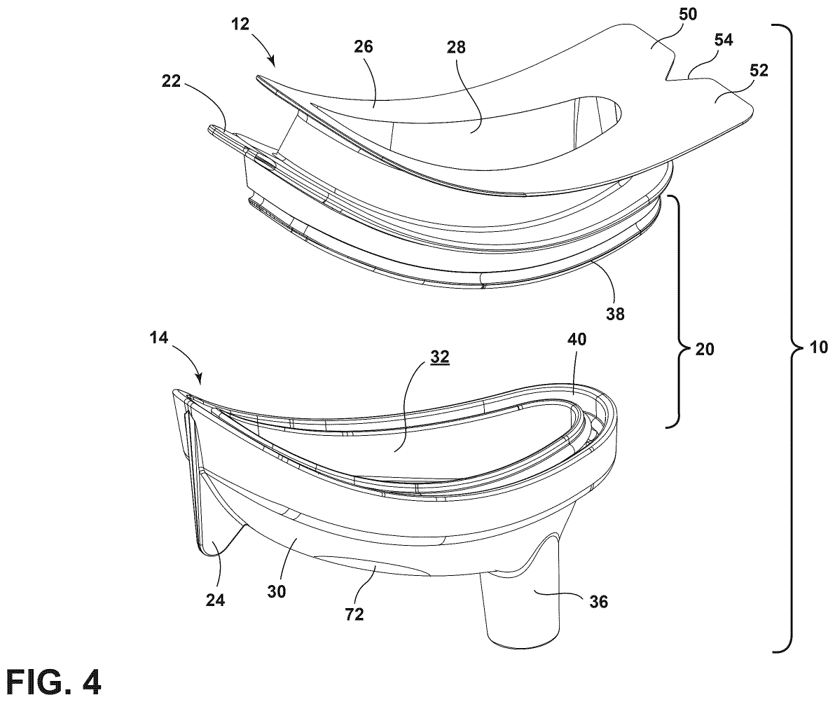

With reference to FIG. 4, the releasable coupling 20 includes a male connector 38 and a female connector 40 configured to receive the male connector 38 for forming a liquid tight seal between the sleeve 12 and the cup 14 and securing the sleeve 12 and cup 14 together. In the illustrated embodiment, the sleeve 12 includes the male connector 38 that is adapted to be removably joined with the corresponding male female connector 40 on the cup 14. In another embodiment, the male connector 38 is disposed on the cup 14 and the female connector 40 is disposed on the sleeve 12.

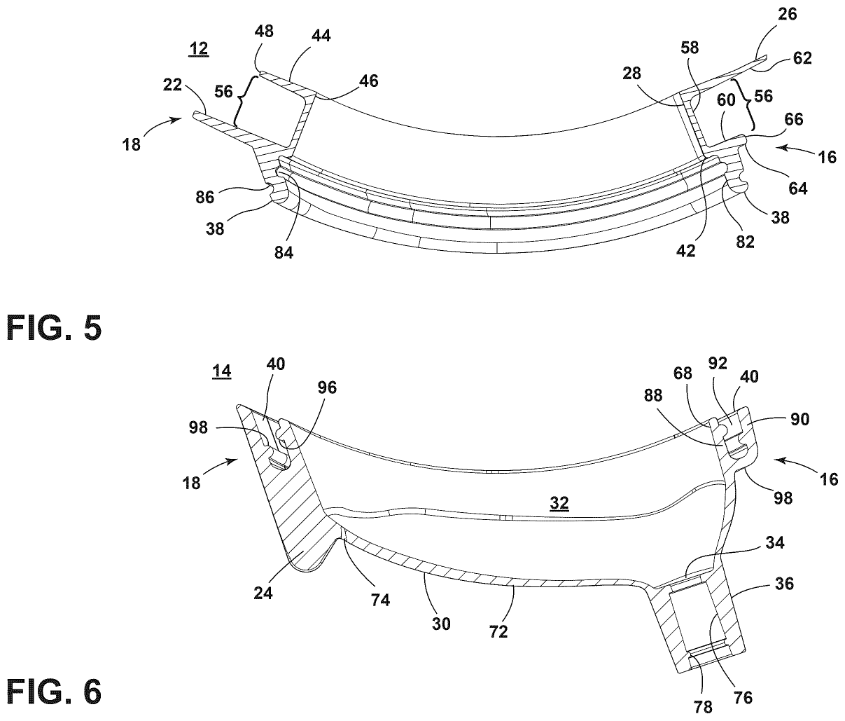

With reference to FIG. 5, which is a sagittal sectional view of the sleeve 12 taken along the sagittal midplane S (FIG. 1), the sleeve 12 includes a second opening or outlet 42, opposite the sleeve inlet 28, through which urine excreted by the user passes into the cup 14. The cup 14 can be coupled with the sleeve at the sleeve outlet 42, with the male connector 38 extending around the periphery of the sleeve outlet 42 and optionally extending distally of the sleeve outlet 42.

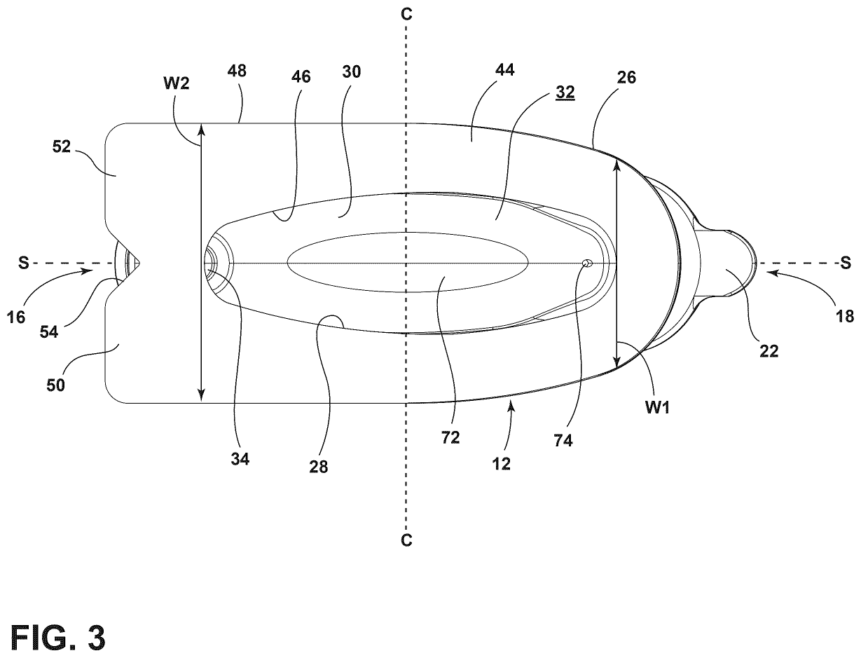

The dimensions of the interface surface 26 can be selected to provide the desired amount of surface area for attaching the sleeve 12 to the user's body through an adhesive. For example, as illustrated in FIGS. 1 and 3, the interface surface 26 can be formed by an annular rim 44 extending outward and away from the sleeve inlet 28 that provides a surface area sufficient to form an adhesive bond with the user's skin to hold the external urine collection device 10 in place. Non-limiting examples of suitable adhesives include a hydrogel, a silicone-based adhesive, and a polyethylene glycol (PEG) hydrogel adhesive.

The sleeve 12 can be contoured to facilitate fitting and adhering the sleeve 12 to the user's body. The dimensions and contour of the sleeve 12 and/or the interface surface 26 can provide a sleeve 12 that is suitable for use by users having different body characteristics. For example, and as best seen in FIG. 3, the interface surface 26 can be contoured to define a curved surface corresponding to a curvature of the user's body adjacent the external urethral orifice, and the interface surface 26 is wider at the posterior end 16 of the device 10 than at the anterior end 18 of the device 10. The interface surface 26 can have a first width W1 at the anterior end 18, for example measured parallel to the coronal plane C at the anterior end of the sleeve inlet 28, and a second width W2 at the posterior end 16, for example measured parallel to the coronal plane C at the posterior end of the sleeve inlet 28, and the second width W2 can be greater than the first width W1 to provide the wider posterior end of the interface surface 26.

The annular rim 44 can have an inner edge 46 forming the inlet 28 and an outer edge 48. The outer edge 48 can have various profiles. In one embodiment, the outer edge 48 is non-circular and is defined in part by a first dorsal flap 50 and a second dorsal flap 52 disposed at a posterior end 16 of the sleeve 12. The dorsal flaps 50, 52 are individually moveable sections of the rim 44 that improve the ability of the interface surface 26 to contour and adhere to the user's body, and as such can take on various shapes, but in general provide more surface area for the rim 44 at the dorsal portion of the device 10. The flaps 50, 52 can be disposed on opposing sides of a sagittal midplane S of the device 10 so that pressure can be applied to each flap 50, 52 individually to contour each flap 50, 52 to the user's body. A notch 54 between the first and second flaps 50, 52 allows individual movement of the flaps 50, 52, and can be generally disposed at the sagittal midplane S of the device 10.

It is noted that dimensions and contour of the sleeve 12 and/or the interface surface 26 shown herein are but one example, and that the dimensions and contour of the sleeve 12 and/or the interface surface 26 can be varied to accommodate users based on their height, weight, age, and/or body measurements. In one example, the sleeve 12 and/or interface surface 26 can be adapted to provide a sleeve 12 in multiple predetermined sizes, such as small, medium, large, and extra-large, etc. to fit a range of physical body types.

The sleeve 12 can be made from a flexible material that facilitates conforming the interface surface 26 to the user's body in order to form a liquid tight seal between the user's body and the sleeve 12. Shaping the interface surface 26 to conform to the user's body also facilitates holding the urinary device 10 in place against the user's body during use. Forming the entire sleeve 12 from a flexible material can also increase the comfort of the user wearing the urinary device 10, particularly when the user is in motion or is sitting. Non-limiting examples of materials suitable for forming the sleeve 12 include silicone or a thermoplastic elastomer. Optionally, only the interface surface 26 is made from a flexible material.

In one aspect, the sleeve 12 can have a finger gap 56 to improve accessibility when adhering the sleeve 12 to the user's body, when attaching the cup 14 to the sleeve 12, and/or when detaching the cup 14 from the sleeve 12. The sleeve 12 includes a side wall 58, with the interface surface 26 at a proximal end of the side wall 58 and a flange 60 at a distal end of the side wall 58. The flange 60 is spaced distally from the interface surface 26 to define the finger gap 56 therebetween. The interface surface 26 and/or flange 60 can extend transversely from the side wall 58, alternately orthogonally from the side wall 58.

The finger gap 56 be dimensioned to fit a caregiver's fingers between the flange 60 and interface surface 26, so that a caregiver can press an underside 62 of the interface surface 26 to adhere the sleeve 12 to the user's body. A caregiver can also press against the underside 62 of the interface surface 26 when detaching the cup 14, to help hold the sleeve 12 in place and limit pulling forces on the user's body.

The flange 60 can extend around the entire periphery of the side wall 58, so that the finger gap 56 extends around the periphery of the sleeve 12. In another embodiment, the flange 60 can extend around only portions of the side wall 58.

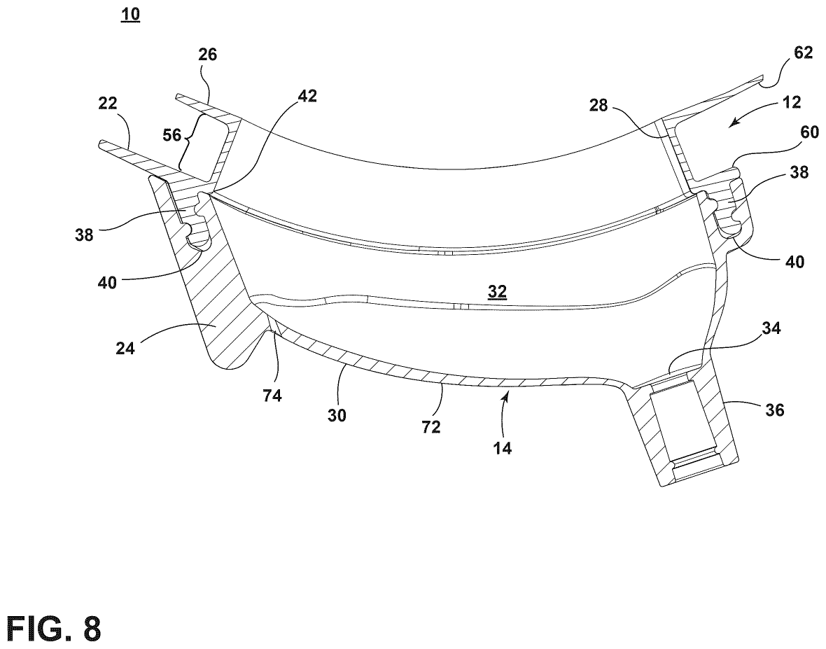

The male connector 38 can extend from an underside 64 of the flange 60 at a position inwardly of an outer edge 66 of the flange 60. The underside 64 of the flange 60 can contact an upper side of the cup 14 when the sleeve 12 and cup 14 are coupled (see FIG. 8).

Referring now to FIG. 6, which is a sagittal sectional view of the cup 14 taken along the sagittal midplane S (FIG. 1), the cup 14 includes a cup inlet 68 through which urine excreted into the sleeve 12 passes into the cup 14 for subsequent draining through the drain connector 36. The drain connector 36 can project from the funnel-shaped wall 30, and is closer to the posterior end 16 than the anterior end 18.

In one embodiment, the drain connector 36 is a press fit connector have a pressure fit connection with the outer diameter of a drain tube (e.g., drain tube 112 in FIG. 9 or drain tube 154 in FIG. 10) for disposal of the drained urine in a collection container (e.g., container 110 in FIG. 9 or drain container 156 in FIG. 10). The pressure fit connection reduces strain on the user's body and on the device 10 by fitting over the outer diameter of the drain tube rather than fitting within an inner diameter of the drain tube. The cup 14 is simply attached to the drain tube by pressing the open end of the drain connector 36 to the end of the drain tube and inserting a length of the drain tube into the drain connector 36 to establish a fluidic connection between the outlet 34 and drain tube. To form a leak-proof seal in communication with the collection chamber 32, the drain connector 36 can have an interior surface 76 that is tapered in the direction of tube insertion and/or has an annular bead 78 thereon that tightly secures the drain tube.

Referring to FIGS. 3 and 6, in at least some embodiments the cup 14 can include a site window 72 in the funnel-shaped wall 30 between the posterior and anterior ends 16, 18 to view the external urethral orifice of the user through the site window 72 without removing the cup 14 from the sleeve 12. The shape, location, and/or dimensions of the site window 72 may be selected to provide the desired viewing characteristics. For example, the site window 72 can comprise a transparent, semitransparent, or translucent region on a side of the wall 30 distal from the user's urethral orifice. The site window 72 can be shaped an oval as illustrated, or can have various other shapes, including, but not limited to, circular or rectangular.

The site window 72 can be formed by selecting a clear material for the cup 14, using a polishing or other finishing process on the wall 30 in the region forming the site window 72, selecting a thickness for the wall 30 in the region forming the site window 72 that is thin enough to see through, or any combination thereof. Non-limiting examples of clear materials for the cup 14 include clear silicone or a transparent thermoplastic elastomer.

With continued reference to FIGS. 3 and 6, the external urine collection device 10 optionally includes an air vent 74 formed in either the sleeve 12 or the cup 14 for venting the collection chamber 32 to facilitate withdrawing urine from the device 10. The air vent 74 can also diminish the degree of suction felt on the user's skin during evacuation of the device 10, increasing the user's comfort level.

In one embodiment, the cup 14 comprises the air vent 74, and optionally the air vent 74 is formed through the cup wall 30. The air vent 74 can be closer to the anterior end 18 than the posterior end 16 of the cup 14. This spaces the air vent 74 away from the drain 36 and prevents leakage of urine through the air vent 74. When worn by a user, the vent 74 is positioned anterior of the user's urethral orifice and the drain 36 is positioned posterior of the user's urethral orifice.

The cup 14 can be made from a material that is flexible to increase the comfort of the user wearing the urinary device 10, particularly when the user is in motion or is sitting. Non-limiting examples of materials suitable for forming the cup 14 include silicone or a thermoplastic elastomer. The cup 14 can be made from the same or different material than the sleeve 12.

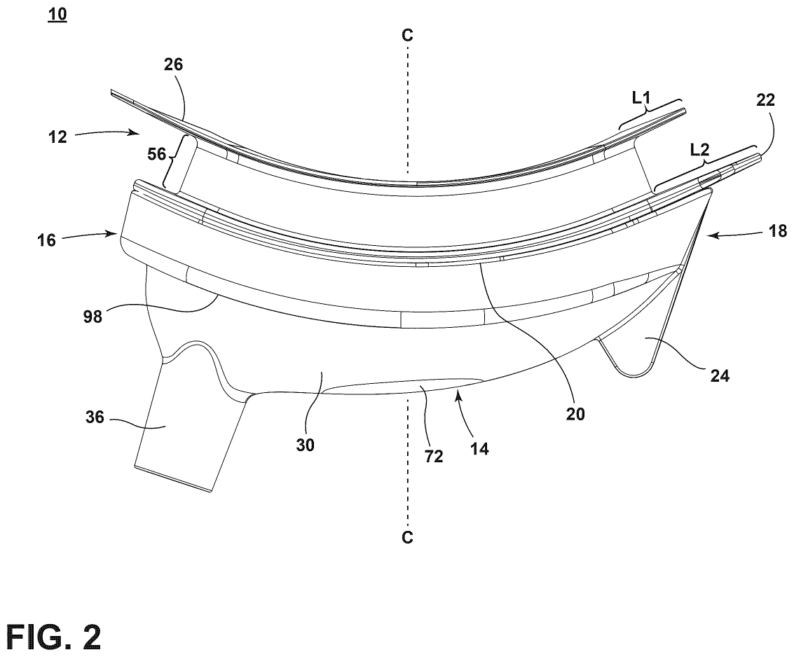

Referring to FIGS. 2 and 7-8, the pull tab 22, 24 can be provided at various locations on the device 10, and in one embodiment the pull tabs 22, 24 are at the anterior end 18 of the device 10, so that the pull tabs pull tab 22, 24 are easily accessible when a user is laying down in a supine position. By gripping the first and second pull tabs 22, 24 and pulling the second pull tab 24 away from the first pull tab 22, the cup 14 can be released from the sleeve 12 while the sleeve 12 remains secured via the adhesive to the external surface of the user. The pull tabs 22, 24 reduce the pulling force transmitted from the sleeve 12 to the user during release of the cup 14, because the first tab 22 can be held to steady the sleeve 12 while pulling only the second tab 24 to start the separation of the cup 14 at the anterior end 18 of the device 10 and continuing to pull until the cup 14 fully separates from the sleeve 12 at the anterior end 18. Optionally, with the same hand holding the first pull tab 22 between thumb and forefinger, the caregiver can apply pressure to the underside 62 of the interface surface 26 with one or more of the other fingers to further steady the sleeve 12.

The second pull tab 24 can extend anteriorly from the cup 14 in a direction along a sagittal midplane S of the cup 14. In one embodiment, the second pull tab 24 lies in the sagittal midplane S such that the second pull tab 24 is pulled in a direction generally along the sagittal plane S of the user to remove the cup 14 from the sleeve 12. This directs the separation force along the sagittal plane S, starting the separation at the anterior end 18 of the device 10 and continuing until the cup 14 separates from the sleeve 12 at the anterior end 18.

As seen in FIG. 7, the first pull tab 22 can extend laterally from opposing sides of the sagittal midplane S. The first pull tab 22 can be generally transverse to the second pull tab 24, and generally transverse to the sagittal midplane S. For example, the first pull tab 22 can be oriented substantially orthogonally to the second pull tab 24. In being “substantially” orthogonal, the pull tabs 22, 24 can be oriented at 90 degrees to each other, alternatively 90 degrees ± up to 5 degrees, ± up to 10 degrees, or ± up to 15 degrees.

The first pull tab 22 can be integrally formed with the flange 60. At the anterior end 18 of the device 10, the first pull tab 22 is spaced from the interface surface 26 in a direction along the vertical axis V of the sleeve 12 to define the finger gap 56 between the interface surface 26 and the first pull tab 22. Alternatively, the pull tab 22 can be separate from the flange 60 forming the finger gap 56.

The second pull tab 24 can be integrally formed with the cup 14. The second pull tab 24 is disposed closer to an anterior end of the cup 14 than a posterior end of the cup 14, and can project from a ventral portion of the cup wall 30. Positioning the pull tab 24 prominently on the device 10 makes the tab easy for the caregiver to locate.

When the cup 14 is assembled with the sleeve 12 as shown in FIG. 2, the first pull tab 22 projects beyond an anterior end of the cup 14. The interface surface 26 can have a first length L1 at an anterior end of the sleeve 12 and the pull tab 22 has a second length L2 at the anterior end of the sleeve 12 that is greater than the first length L1 such that the pull tab 22 projects beyond the interface surface 26. Said lengths L1, L2 can, for example, be measured from the side wall 58 of the sleeve 12 at the anterior end of the sleeve 12. Positioning the pull tabs 22 prominently on the device 10 makes the tab easy for the caregiver to locate.

Referring to FIGS. 5-6, various embodiments of the male and female connectors 38, 40 are possible. In one embodiment, the male connector 38 includes a leg 82 having an inner seal recess 84 on a side adjacent the sleeve outlet 42 and an outer seal recess 86 on the opposing side of the leg 82.

The sleeve 12 is illustrated in FIG. 5 as having the leg 82 of the male connector 38 extending from a lower side of the flange 60 at the distal end of the sleeve side wall 58 and is outward of the side wall 58. In another embodiment (not illustrated), the male connector 38 is separate from the flange 60 and/or the leg 82 is inward of the side wall 58 or aligned with the side wall 58.

The female connector 40 includes an inner flange 88 adjacent the collection chamber 32 and an opposing outer flange 90. The inner and outer flanges 88, 90 together define a channel 92 that is adapted to receive the male connector 38 therein for forming the liquid tight seal between the sleeve 12 and the cup 14.

As illustrated in FIG. 6, the inner flange 88 defines the cup inlet 68 and forms an upper portion of the cup wall 30. In another embodiment (not illustrated), the inner flange 88 is separate from the cup wall 30 and/or the inlet 68 into the cup 14 is defined by a wall that is separate from the female connector 40. In one such configuration, the inner flange 88 forming the female connector 40 can extend around the periphery of the separate cup wall 30, thus forming a liquid tight seal around the periphery of the separate cup wall.

Still referring to FIG. 6, the outer flange 90 can be formed by an outer wall that is joined to or otherwise coupled with the cup wall 30 by at a shoulder 98. The shoulder 98 can extend around the entire periphery of the cup 14 or only portions of the cup 14. Pressure can be applied to the shoulder 98 when attaching the cup 14 to the sleeve 12 to ensure complete insertion of the female connector 40 into the male connector 38 around the entire cup inlet 68.

The inner and outer flanges 88, 90 each include a rib 94, 96, respectively, extending into the channel 92. The ribs 94, 96 can be configured to facilitate forming the liquid tight seal with the male connector 38. The inner and outer seal recesses 84, 86 are configured to mate with the ribs 94, 96, respectively, to form the liquid tight seal between the sleeve 12 and the cup 14.

The ribs 94, 96 can extend around the entire periphery of the channel 92 or only portions of the channel 92. Optionally, only one of the ribs 94, 96 is disposed within the channel 92. Further optionally, one or both of the inner and outer flanges 88, 90 include multiple ribs 94, 96 to facilitate forming a liquid tight seal and securing the sleeve 12 and cup 14 together.

The inner and/or outer seal recesses 84, 86 can extend around an entire periphery of the cup inlet 68 or only a portion thereof. Optionally, the leg 82 includes a seal recess 84, 86 on a single side of the leg 82 corresponding to the location of a mating rib 94, 96 on one of the inner or outer cup flanges 88, 90. Further optionally, the leg 82 includes multiple seal recesses 84, 86 on one or both sides of the leg 82 based on the location of mating ribs 94, 96 on one or both of the cup flanges 88, 90.

In another embodiment, the location of the seal recesses 84, 86 and the ribs 94, 96 can be reversed. The ribs 94, 96 can be carried by the sleeve leg 82 and the mating seal recesses 84, 86 can be carried by the cup flanges 88, 90 in order to form a liquid tight seal between the sleeve 12 and the cup 14.

The male and female connectors 38, 40 are configured such that as the leg 82 of the male connector 38 is inserted into the channel 92 of the female connector 40, the inner and outer flanges 88, 90 resiliently flex away from one another to receive the leg 82. Optionally, the female connector 40 can be configured such that only one of the inner or outer flanges 88, 90 is resilient and flexes away from the other flange when the leg 82 is inserted into the channel 92. The ribs 94, 96 are adapted to engage the seal recesses 84, 86 to form a liquid tight seal between the sleeve 12 and cup 14. The formation of a liquid tight seal between the sleeve 12 and the user's body and the formation of a liquid tight seal between the sleeve 12 and the cup 14 provides a pathway through which urine excreted by the user can be collected and then exit the device 10 through the drain 36 in a sealed, non-leaking manner.

Referring now to FIGS. 8-10, the assembly and use of the external urine collection device 10 as part of an external urine collection system is illustrated and described. The external urine collection device 10 is intended to be worn by a user as an external catheter to collect and drain urine excreted by the user away from the user's body. The external urine collection device 10 may be applied to the user by the user themself or by another caregiver.

To fit the external urine collection device 10 onto the user, the sleeve 12 is attached around the user's external urethral orifice by an adhesive to form a liquid tight seal between the user's body and the sleeve 12. The sleeve 12 can be separated from the cup 14 prior to attaching the sleeve 12 to facilitate aligning the sleeve 12 around the user's urethral orifice. When the cup 14 is separated from the sleeve 12, the user's body can be viewed through the open sleeve inlet 28 and sleeve outlet 42 as the sleeve 12 is being fitted to the user. Optionally, the sleeve 12 can be attached to the user's body with the cup 14 coupled to the sleeve 12.

The adhesive is adapted to form a bond between the user's body and the interface surface 26 of the sleeve 12 to hold the sleeve 12 in position against the user's body and to provide the liquid tight seal between the sleeve 12 and the user's body. The adhesive can be pre-applied to the interface surface 26 or applied to the interface surface 26 and/or the user's body at the time of attachment. In one example, the adhesive is pre-applied to the interface surface 26 and is covered with a release liner that is removed prior to attaching the sleeve 12 to the user's body.

The adhesive may be any adhesive that is suitable for use on human skin, is non-toxic, and is capable of providing a liquid tight seal and retaining the sleeve 12 in position on the user's body for a predetermined period of time. The adhesive can be selected based on the ease in which the adhesive can be removed from the user without excessive discomfort and/or pain to the user. The adhesive can be configured to maintain the liquid tight seal and retain the sleeve 12 in place for a minimum period of time, such as 12 hours or 24 hours, for example.

One example of a suitable adhesive is a PEG hydrogel. Many types of PEG hydrogels are non-toxic, safe on sensitive skin, and can absorb enough moisture to form a liquid tight seal with the sleeve 12. Another example is a “smart” or environmentally-sensitive hydrogel that exhibits swelling and de-swelling behavior that is temperature sensitive. A temperature sensitive adhesive includes a lower critical solution temperature (LCST), above which the adhesive hydrogel de-swells and collapses. This type of temperature sensitive hydrogel can be removed by increasing the temperature of the adhesive to a temperature above the LCST. Yet another example of a suitable adhesive is a silicone gel adhesive, such as those conventionally used in wound care.

The formation of the liquid tight seal between the sleeve 12 and the user's body is facilitated by the 3-dimensional contours of the sleeve 12 and/or the materials used to form the sleeve 12. The sleeve 12, including the interface surface 26, can be configured to conform to the contours of the user's body adjacent the external urethral orifice to facilitate forming the liquid tight seal between the sleeve 12 and the user's body. Forming the sleeve 12 from a flexible material facilitates bending and/or flexing the sleeve 12 to provide a good fit of the sleeve 12 onto the user. Optionally, the sleeve 12 can be provided in one of several sizes, such as small, medium, large, etc. to facilitate providing a good fit between the sleeve 12 and the user's body. A sleeve 12 in the size which provides the best fit to the user can be selected to facilitate forming the liquid tight seal around the user's external urethral orifice.

Prior to attaching the sleeve 12 to the user's body, the area around the external urethral orifice is optionally prepped to facilitate forming the liquid tight seal and forming a good attachment between the adhesive and the user's skin. Preparing the user's body can include cleaning the area around the external urethral orifice using water, soap, and/or alcohol and optionally drying the cleaned area. Preparing the area can also include shaving body hair in the area around the external urethral orifice to facilitate bonding between the user's skin and the adhesive attaching the sleeve 12.

If the cup 14 is not already coupled to the sleeve 12, subsequent to attaching the sleeve 12 to the user's body, the cup 14 can be fitted with the sleeve 12 to form a liquid tight seal between the sleeve 12 and the cup 14. To couple the sleeve 12 and the cup 14, the male connector 38 on the cup 14 is aligned with the female connector 40 on the sleeve 12 and the cup 14 is pressed against the sleeve 12. When coupling the cup 14 with the sleeve 12, the caregiver can insert one or more fingers into the finger gap 56 between the flange 60 and interface surface 26 and press on the underside 62 of interface surface 26 to hold the sleeve 12 generally steady. The caregiver can also press on the shoulder 98 of the cup 14 to ensure complete insertion of the female connector 40 into the male connector 38 around the entire cup inlet 68.

Subsequent to the attachment of the device 10 to the user, the site can be visually inspected without detaching the cup 14 through the site window 72. The site window 72 allows the area encompassed by the device 10 to be visually inspected without having to remove the cup 14. In addition, leaving the cup 14 in place also avoids discomfort to the user associated with removing the cup 14.

Subsequent to the attachment of the device 10 to the user, the cup 14 can be removed without detaching the sleeve 12 to allow for access to the site and/or cleaning of the area encompassed by the sleeve 12. The cup 14 can be removed by withdrawing the cup 14 and unseating the male connector 38 from the female connector 40. When uncoupling the cup 14 from the sleeve 12, the caregiver can grip the first pull tab 22 in one hand and grip the second pull tab 24 in the other hand, and pull the second pull tab 24 away from the first pull tab 22. The caregiver can also insert one or more fingers of the hand holding the first pull tab 22 into the finger gap 56 between the flange 60 and interface surface 26 and press on the underside 62 of interface surface 26 to hold the sleeve 12 steady. The pull tabs 22, 24 reduce the pulling force transmitted from the sleeve 12 to the user during release of the cup 14, and the cup 14 can reliably be removed without unseating the adhesively bonded sleeve 12 from the user's body. Leaving the bonded sleeve 12 in place allows the area to be accessed and/or cleaned quickly and easily without having to remove and reapply adhesives. In addition, leaving the adhesively bonded sleeve 12 in place also avoids discomfort to the user associated with removing an adhesive from the skin. The separated cup 14 can also be cleaned and optionally replaced prior to re-coupling the cup 14 with the sleeve 12. The cup 14 can be re-coupled with the sleeve 12 through the male and female connectors 38, 40 in the same manner as described above.

The adhesive, and thus the sleeve 12, can be configured to be removed from the user's body within a predetermined period of time and optionally replaced with a new sleeve 12. The predetermined period of time can be, for example, at least 12 hours, alternatively between 12 and 24 hours. The manner in which the sleeve 12 is separated from the user's body can be based on the type of adhesive used to attach the sleeve 12.

Referring now to FIG. 9, the external urine collection device 10 can be coupled to a collection container 110 to as part of an external urine collection system 100 to collect the urine excreted by the user for subsequent disposal. The device 10 can be coupled to the container 110 prior to or subsequent to attaching the device 10 to the user's body. The device 10 can be connected to the container 110 through a drain tube 112 that is coupled to the drain connector 36. Urine collected in the device 10 can exit through the drain connector 36 and then flow through the drain tube 112 and into the container 110. The collection container 110 can be any suitable type of container, such as a leg bag or bedside bag, which is capable of storing urine until it can be disposed of. Optionally, the drain tube 112 and/or the container 110 includes a one-way seal to prevent urine from back-flowing into the device 10.

The flow of urine from the device 10 to the container 110 can be assisted by gravity. Optionally, the container 110 is coupled to a pump 114 by a tube 116. The pump 114 can apply negative pressure to the device 10 to facilitate draining urine from the cup 14 through the drain connector 36 and into the container 110. In one example, the pump 114 is a vacuum pump. The vacuum pump can be an individual unit or a part of an in-house suction system that can be coupled to the container 110. For patients who are mobile, gravity may be sufficient to drain urine from the device 10 into the container 110 at a desired rate. However, for users who are lying down and/or are not mobile, the pump 114 can be used to facilitate draining the urine from the device 10 at a desired rate. In addition, the pump 114 can facilitate removing excess moisture that would otherwise remain within the device 10.

Referring now to FIG. 10, another example of an external urine collection system 150 is illustrated. The system 150 is similar to the system 100 in structure, function, and operation, with several exceptions. For example, the manner in which negative pressure is applied to the device 10 differs. The system 150 includes a suction device 152 coupled to the device 10 by a drain tube 154. The suction device 152 can be a manual or powered device capable of applying negative pressure to facilitate draining urine from the device 10. The suction device 152 is coupled with a waste tube 158 for transporting the drained urine into a collection container 156 for storage and subsequent disposal. In one example, the suction device 152 can be a manual device, such as a squeeze pump or a bellows pump. In another example, the suction device 152 is a peristaltic pump.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims. Any reference to claim elements in the singular, for example, using the articles “a,” “an,” “the” or “said,” is not to be construed as limiting the element to the singular. Any reference to claim elements as “at least one of X, Y and Z” is meant to include any one of X, Y or Z individually, and any combination of X, Y and Z, for example, X, Y, Z; X, Y; X, Z; and Y, Z.

What is claimed is:

1. An external urine collection device for urine excreted by a user through the user's external urethral orifice, the external urine collection device comprising:
a posterior end and an anterior end defined with respect to an anatomical orientation of the collection device when the collection device is worn by the user, with the posterior end positioned posterior of an external urethral orifice of the user and the anterior end positioned anterior of the external urethral orifice of the user when the collection device is worn by the user;
a sleeve having a sleeve outlet and an interface surface defining a sleeve inlet, the interface surface configured to be secured via an adhesive to an external surface of the user and to surround an external urethral orifice of the user;
a cup coupled with the sleeve at the sleeve outlet, the cup including a collection chamber and a urine outlet;
a releasable coupling between the cup and the sleeve, the releasable coupling forming a liquid tight seal for the flow of urine through the sleeve and into the cup;
a first pull tab on the sleeve at the anterior end of the collection device; and
a second pull tab on the cup at the anterior end of the collection device;
wherein the cup is releasable from the sleeve while the sleeve remains secured via the adhesive to the external surface of the user by a caregiver gripping the first and second pull tabs and pulling the second pull tab away from the first pull tab while the first pull tab remains proximate the user;
whereby the first and second pull tabs reduce the pulling force transmitted from the sleeve to the user during release of the cup by the caregiver gripping the first pull tab to steady the sleeve relative to the user and pulling the second tab away from the sleeve while the sleeve is held generally steady by the caregiver via the first pull tab.

2. The external urine collection device of claim 1, wherein the second pull tab extends anteriorly from the cup in a direction along a sagittal midplane of the cup.

3. The external urine collection device of claim 2, wherein the first pull tab is transverse to the second pull tab.

4. The external urine collection device of claim 1, wherein the collection device comprises a sagittal midplane configured to lie substantially within or parallel to a sagittal plane of the user when the collection device is worn by the user, wherein the second pull tab lies in the sagittal midplane such that the second pull tab is pulled in a direction generally along the sagittal plane of the user to remove the cup from the sleeve.

5. The external urine collection device of claim 4, wherein the first pull tab is transverse to the second pull tab.

6. The external urine collection device of claim 4, wherein the first pull tab extends laterally from opposing sides of the sagittal midplane.

7. The external urine collection device of claim 4, wherein the sleeve comprises a finger gap, wherein the first pull tab is spaced from the interface surface in a direction along a vertical axis of the sleeve to define the finger gap between the interface surface and the first pull tab.

8. The external urine collection device of claim 1, wherein the sleeve comprises a finger gap, wherein the sleeve comprises a side wall, with the interface surface at a proximal end of the side wall and a flange at a distal end of the side wall, wherein the flange extends transversely from the side wall and the flange is spaced distally from the interface surface to define the finger gap therebetween.

9. The external urine collection device of claim 8, wherein the first pull tab is integrally formed with the flange.

10. The external urine collection device of claim 1, wherein the sleeve comprises a flange spaced from the interface surface to define a finger gap therebetween, wherein the finger gap is dimensioned to fit a caregiver's fingers between the flange and interface surface.

11. The external urine collection device of claim 1, wherein the interface surface has a first length at the anterior end of the sleeve and the first pull tab has a second length at the anterior end of the sleeve that is greater than the first length such that the first pull tab projects beyond the interface surface.

12. The external urine collection device of claim 1, wherein the first pull tab projects beyond an anterior end of the cup and wherein the second pull tab is disposed closer to an anterior end of the cup than a posterior end of the cup and projects distally from the cup.

13. The external urine collection device of claim 1, wherein the first pull tab is oriented substantially orthogonally to the second pull tab.

14. The external urine collection device of claim 1, wherein the cup comprises:
a funnel-shaped wall defining the collection chamber, the urine outlet comprising an opening in the funnel-shaped wall; and
a drain connector through which urine can exit the collection chamber via the urine outlet;
wherein the drain connector projects from the funnel-shaped wall and is closer to the posterior end than the anterior end; and
wherein the drain connector comprises a press fit connector configured to receive an outer diameter of a drain tube.

15. The external urine collection device of claim 1, wherein the cup comprises:
a funnel-shaped wall defining the collection chamber; and
a site window in the funnel-shaped wall between the posterior and anterior ends, wherein the external urethral orifice of the user can be viewed through the site window without removing the cup from the sleeve.

16. The external urine collection device of claim 1, wherein the interface surface comprises an annular rim having an inner edge forming the sleeve inlet and a non-circular outer edge defined in part by a first dorsal flap and a second dorsal flap disposed at a posterior end of the sleeve, wherein the first and second dorsal flaps are disposed on opposing sides of a sagittal midplane of the collection device.

17. The external urine collection device of claim 1, wherein the interface surface is contoured to define a curved surface corresponding to a curvature of the user's body adjacent the external urethral orifice, wherein the interface surface is wider at the posterior end than at the anterior end.

18. The external urine collection device of claim 1, wherein the releasable coupling comprises:
a male connector disposed on one of the sleeve and the cup; and
a female connector disposed on the other one of the sleeve and the cup;
wherein the female connector is configured to receive the male connector.

19. An external urine collection device for urine excreted by a user through the user's external urethral orifice, the external urine collection device comprising:
a sleeve having a sleeve outlet and an interface surface defining a sleeve inlet, the interface surface configured to be secured via an adhesive to an external surface of the user and to surround an external urethral orifice of the user;
a cup removably coupled with the sleeve, the cup including a collection chamber and a urine outlet;
a first pull tab on the sleeve at a first end of the collection device; and
a second pull tab on the cup at the first end of the collection device;
wherein the cup is releasable from the sleeve while the sleeve remains secured via the adhesive to the external surface of the user by a caregiver gripping the first and second pull tabs and pulling the second pull tab away from the first pull tab while the first pull tab remains proximate the user.

20. An external urine collection device for urine excreted by a user through the user's external urethral orifice, the external urine collection device comprising:
a posterior end and an anterior end defined with respect to an anatomical orientation of the collection device when the collection device is worn by the user, with the posterior end positioned posterior of an external urethral orifice of the user and the anterior end positioned anterior of the external urethral orifice of the user when the collection device is worn by the user;
a sagittal midplane configured to lie substantially within or parallel to a sagittal plane of the user when the collection device is worn by the user;
a sleeve comprising:
a side wall;
a sleeve outlet;
an interface surface defining a sleeve inlet, the interface surface configured to be secured via an adhesive to an external surface of the user and to surround an external urethral orifice of the user, the interface surface at a proximal end of the side wall;
a flange at a distal end of the side wall, wherein the flange extends transversely from the side wall and the flange is spaced distally from the interface surface to define a finger gap therebetween;
a cup coupled with the sleeve at the sleeve outlet, the cup including a collection chamber and a urine outlet;
a releasable coupling between the cup and the sleeve, the releasable coupling forming a liquid tight seal for the flow of urine through the sleeve and into the cup;
a first pull tab on the sleeve at the anterior end of the collection device; and
a second pull tab on the cup at the anterior end of the collection device;
wherein the cup is releasable from the sleeve while the sleeve remains secured via the adhesive to the external surface of the user by a caregiver gripping the first and second pull tabs and pulling the second pull tab away from the first pull tab while the first pull tab remains proximate the user;
wherein the second pull tab lies in the sagittal midplane such that the second pull tab is pulled in a direction generally along the sagittal plane of the user to remove the cup from the sleeve; and
whereby the first and second pull tabs reduce the pulling force transmitted from the sleeve to the user during release of the cup by the caregiver gripping the first pull tab to steady the sleeve relative to the user and pulling the second tab away from the sleeve in a direction generally along the sagittal plane of the user while the sleeve is held generally steady by the caregiver via the first pull tab.
